(19) United States
(12) Patent Application Publication
(10) Pub. No.: US 2021/0198152 A1
(43) Pub. Date: Jul. 1, 2021

(54) CERAMIC PARTICLE AND METHOD FOR PRODUCING THE SAME

(71) Applicant: METAL INDUSTRIES RESEARCH & DEVELOPMENT CENTRE, Kaohsiung City (TW)

(72) Inventors: Chun-Chieh Tseng, Kaohsiung City (TW); Yen-Hao Chang, Yuanlin City (TW); Yue-Jun Wang, New Taipei City (TW); Tung-Lin Tsai, Tainan City (TW); Ying-Cheng Lu, Kaohsiung City (TW); Chiu-Feng Lin, Pingtung City (TW)

(21) Appl. No.: 16/727,145

(22) Filed: Dec. 26, 2019

(51) Int. Cl. C04B 35/043; C04B 35/632; C04B 35/628; C04B 35/515

(52) U.S. Cl. CPC C04B 35/043; C04B 35/632; C04B 35/62842; C04B 2235/5436; C04B 2235/3206; C04B 2235/401; C04B 2235/3212; C04B 35/515

(57) ABSTRACT

A ceramic particle includes a core and a modification layer. The core is made of magnesium or a magnesium alloy. The core has a diameter of 30-100 μm. The modification layer covers an outer surface of the core. The modification layer includes calcium and phosphorus. A method for producing a ceramic particle includes providing a core made of magnesium or a magnesium alloy and having a diameter of 30-100 μm. A calcium salt and a phosphorus salt are dissolved in a solvent. A chelating agent is added into the solvent to form a modifying solution. The core is added into the modifying solution to form a modification layer on an outer surface of the core in a temperature range of 5-40° C. The modification layer includes calcium and phosphorus.

### BACKGROUND OF THE INVENTION

### 1. Field of the Invention

The present invention relates to a ceramic particle and, more particularly, to a ceramic particle with modified qualities. The present invention also relates to a method for producing the ceramic particle.

### 2. Description of the Related Art

Magnesium is light and has characteristics, such as a density and an elastic coefficient, similar to a periosteum. Furthermore, magnesium has excellent mechanical characteristics and is a biodegradable material. Thus, magnesium alloys obtained from mixing magnesium and other metals are potential biomedical materials for substituting titanic alloys and stainless steel.

However, magnesium alloys have a poor anti-corrosion effect and degrade quickly in organisms. Thus, a surface of a conventional implant made of a magnesium alloy is generally covered by an anti-corrosion layer made of hydroxyapatite to prevent rapid degradation of the conventional implant after being implanted into an organism. However, the mechanical properties of the anti-corrosion layer are quite different from those of the conventional implant, such that when the anti-corrosion layer is broken, the whole piece of the anti-corrosion layer is apt to peel off during use.

Thus, it is necessary to mitigate and/or obviate the conventional drawbacks.

### SUMMARY OF THE INVENTION

To solve the above drawbacks, an objective of the present invention is to provide a ceramic particle for forming an implant, and the implant has an excellent anti-corrosion effect without the need of formation of an extra anti-corrosion layer.

Another objective of the present invention is to provide a method for producing the above-mentioned ceramic particle.

A ceramic particle according to the present includes a core and a modification layer. The core is made of magnesium or a magnesium alloy. The core has a diameter of 30-100 μm. The modification layer covers an outer surface of the core. The modification layer includes calcium and phosphorus.

Thus, in the ceramic particle according to the present invention, by covering the outer surface of the core with the modification layer, after an implant made of the ceramic particle is implanted into an organism, abnormal reaction is less likely to occur, and the bone cells can be stimulated to crawl onto the implant, such that the implant can tightly bond with the bone. These are the effects of the present invention. Furthermore, since the ceramic particle 1 includes the modification layer, an excellent anti-corrosion effect can be obtained without the need of forming an extra anti-corrosion layer on an outer surface of the implant. This avoids troublesome procedures for forming the anti-corrosion layer. Furthermore, since the implant is free of the anti-corrosion layer, peeling of the whole piece of the anti-corrosion layer during use can be avoided. Thus, the present invention provides enhanced utility of the implant.

In an example of the ceramic particle, the modification layer has a thickness of 0.1-5 μm. Thus, the outer surface of the core has the modification layer of a sufficient thickness to provide the ceramic particle with excellent properties, such as excellent biocompatibility and osteoinduction.

In an example of the ceramic particle, the mole ratio of calcium to phosphorus in the modification layer is in a range of 1.0-1.8. The modification layer is formed of calcium monohydrogen phosphate, hydroxyapatite, or tricalcium diphosphate. Thus, the ceramic particle has excellent properties, such as excellent biocompatibility and osteoinduction.

A method for producing a ceramic particle according to the present invention includes providing a core made of magnesium or a magnesium alloy, wherein the core has a diameter of 30-100 μm; dissolving a calcium salt and a phosphorus salt in a solvent, and adding a chelating agent into the solvent to form a modifying solution; and adding the core into the modifying solution to form a modification layer on an outer surface of the core in a temperature range of 5-40° C., wherein the modification layer includes calcium and phosphorus.

Thus, the modification layer can be uniformly formed on the outer surface of the core by controlling the temperature, such that after an implant made of the ceramic particle is implanted into an organism, abnormal reaction is less likely to occur, and the bone cells can be stimulated to crawl onto the implant, such that the implant can tightly bond with the bone. These are the effects of the present invention.

In an example of the method for producing the ceramic particle, the calcium salt is calcium nitrate, calcium phosphate, or calcium sulfate. The phosphorus salt is potassium dihydrogen phosphate, sodium dihydrogen phosphate, or magnesium phosphate. The chelating agent is edetate disoium or ethylenediamine Thus, by selecting a proper calcium salt and a proper phosphate and cooperating with a proper chelating agent, a modification layer formed by a specific compound can be formed on the outer surface of the core, such that the ceramic particle has excellent properties, such as excellent biocompatibility and osteoinduction.

The present invention will become clearer in light of the following detailed description of illustrative embodiments of this invention described in connection with the drawings.

### BRIEF DESCRIPTION OF THE DRAWINGS

FIG. 1 is a cross sectional view of a ceramic particle of an embodiment according to the present invention.

FIG. 2a is an SEM image of cores of ceramic particles obtained in test A.

FIG. 2b is an SEM image of ceramic particles obtained in test A.

FIG. 2c is an enlarged view of a region R in FIG. 2b.

FIG. 3 is a diagram illustrating a hydrogen yield of each sample in test B.

### DETAILED DESCRIPTION OF THE INVENTION

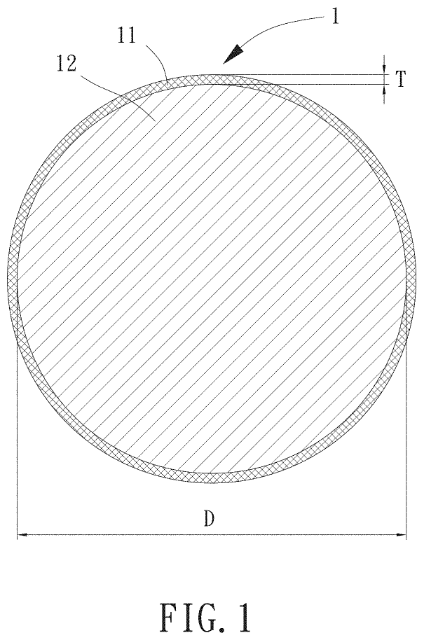

With reference to FIG. 1, a ceramic particle 1 of an embodiment according to the present includes a core 11 and a modification layer 12. The modification layer 12 covers an outer surface of the core 11. Specifically, the core 11 is made of magnesium or a magnesium alloy. In an example, the magnesium alloy includes calcium, zinc, manganese, cerium, thorium, or zirconium. Thus, the mechanical properties, such as the yield strength, elongation, etc., of the core 11 can be improved.

Furthermore, the core 11 can be formed by gas atomization. A nozzle is used to turn molten metal into droplets, which cools down and solidifies into the core 11. Thus, the resultant core 11 has a higher sphericity. In this embodiment, the core has a diameter D of 30-100 μm.

The modification layer includes calcium (Ca) and phosphorus (P). Preferably, the mole ratio of calcium to phosphorus (Ca/P ratio) in the modification layer 12 is in a range of 1.0-1.8. For example, the modification layer 12 is formed of a compound including calcium and phosphorus, such as calcium monohydrogen phosphate (CaHPO₄), hydroxyapatite (Ca₅(PO₄)₃(OH)), or tricalcium diphosphate (Ca₃(PO₄)₂). With the properties, such as biocompatibility and osteoinduction, of the compounds including calcium and phosphorus, after the implant made of the ceramic particle 1 is implanted into an organism, abnormal reaction is less likely to occur, and the bone cells can be stimulated to crawl onto the implant, such that the implant can tightly bond with the bone.

In this embodiment, a worker can dissolve a calcium sulfate and a phosphate in a solvent, and a chelating agent is added into the solvent to form a modifying solution. Both the calcium concentration and the phosphorous concentration are in a range of 0.05-0.5 M. For example, the calcium salt can be calcium nitrate (Ca(NO₃)₂), calcium phosphate (Ca₃(PO₄)₂), or calcium sulfate (CaSO₄). The phosphorus salt can be potassium dihydrogen phosphate (KH₂PO₄), sodium dihydrogen phosphate (NaH₂PO₄), or magnesium phosphate (Mg₃(PO₄)₂). The chelating agent can be edetate disoium (C₁₀H₁₄N₂Na₂O₈) or ethylenediamine (C₂H₄(NH₂)₂). The chelating agent can be water. Furthermore, a worker can select a specific calcium salt and a specific phosphorus salt and can adjust the ratio of the calcium salt to the phosphorus salt to a specific value, such that the specific compound including calcium and phosphorus can form the modification layer 12.

Then, the core 11 is added into the modifying solution and is stirred at 5-40° C. for 5-30 minutes, such that the modification layer 12 can be formed on an outer surface of the core 11, obtaining the ceramic particle 1. The thickness T of the modification layer 12 can be in a range of 0.1-5 μm.

It is worth noting that the ceramic particles 1 of this embodiment can be used to form an implant (such as a bone nail, a dental implant or a bone plate) by injection molding, powder metallurgy, 3D printing, or compression molding. The implant can be implanted into an organism and, thus, can be used in reconstruction of a tooth, a bone, or a joint of the organism. Since the implant is made of the ceramic particles 1 of this embodiment, an excellent anti-corrosion effect can be obtained without the need of forming an extra anti-corrosion layer on an outer surface of the implant.

To prove that the method according to the present invention can be used to produce the ceramic particle 1 and that a metal ingot formed by the ceramic particles 1 indeed has a better anti-corrosion effect, the following tests are carried out.

Test (A): Images Taken by a Scanned Electron Microscope (SEM)

This test uses a magnesium alloy particle produced from gas atomization as the core 11. The core 11 is added into the modifying solution (containing calcium nitrate, potassium dihydrogen phosphate, and edetate disoium, and the pH value is adjusted to be in a range of 4-6) to form the modification layer 12 on the outer surface of the core 11, and the ceramic particle 1 is obtained after filtration and draying.

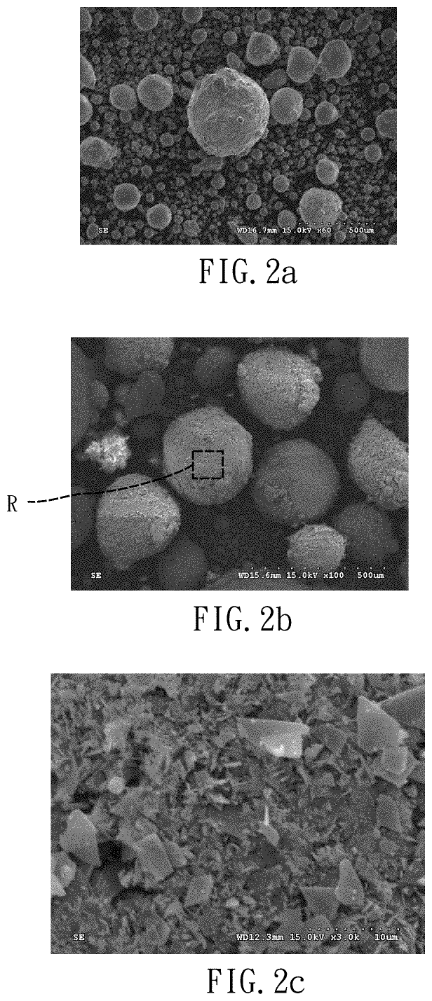

Next, a scanned electron microscope is used to take images of the core 11 and the ceramic particle 1, and the results are shown in FIGS. 2a, 2b, and 2c. As can be seen from the figures, the roughness of the outer surface of the ceramic particle 1 is different from the roughness of the outer surface of the core 11, which is an indication that the modification layer 12 is formed on the outer surface of the core 11.

Test (B): Evaluation of the Anti-Corrosion Capability

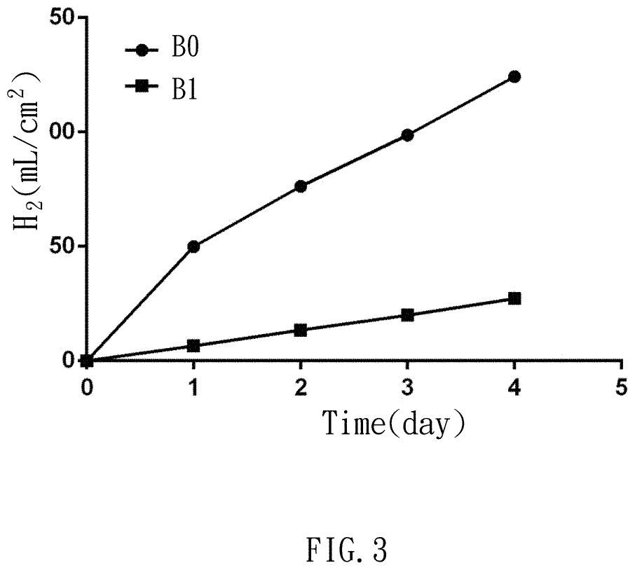

This test uses a metal ingot formed by the ceramic particles 1 as group B1 and uses a metal ingot formed by the cores 11 as group B0. The metal ingots of groups B0 and B1 are placed in a simulated body fluid (SBF), and the hydrogen yield is calculated every day to thereby evaluate the anti-corrosion capability of the metal ingots of groups B0 and B1. The results are shown in FIG. 3. Since the hydrogen yield of the metal ingot formed of the ceramic particle 1 is significantly lower than that of the metal ingot formed of the core 11, the metal ingot formed of the ceramic particle 1 has better anti-corrosion capability.

In view of the foregoing, in the ceramic particle 1 according to the present invention, by covering the outer surface of the core 11 with the modification layer 12, after an implant made of the ceramic particle 1 is implanted into an organism, abnormal reaction is less likely to occur, and the bone cells can be stimulated to crawl onto the implant, such that the implant can tightly bond with the bone. These are the effects of the present invention.

Furthermore, since the ceramic particle 1 includes the modification layer 12, an excellent anti-corrosion effect can be obtained without the need of forming an extra anti-corrosion layer on an outer surface of the implant. This avoids troublesome procedures for forming the anti-corrosion layer. Furthermore, since the implant is free of the anti-corrosion layer, peeling of the whole piece of the anti-corrosion layer during use can be avoided. Thus, the present invention provides enhanced utility of the implant.

Furthermore, in the method for producing the ceramic particle 1 according to the present invention, the modification layer 12 can be uniformly formed on the outer surface of the core 12 by controlling the temperature, such that after an implant made of the ceramic particle 1 is implanted into an organism, abnormal reaction is less likely to occur, and the bone cells can be stimulated to crawl onto the implant, such that the implant can tightly bond with the bone. These are the effects of the present invention.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A ceramic particle comprising:
a core made of magnesium or a magnesium alloy, wherein the core has a diameter of 30-100 μm; and
a modification layer covering an outer surface of the core, wherein the modification layer includes calcium and phosphorus.

2. The ceramic particle as claimed in claim 1, wherein the modification layer has a thickness of 0.1-5 μm.

3. The ceramic particle as claimed in claim 1, wherein a mole ratio of calcium to phosphorus in the modification layer is in a range of 1.0-1.8.

4. The ceramic particle as claimed in claim 3, wherein the modification layer is formed of calcium monohydrogen phosphate, hydroxyapatite, or tricalcium diphosphate.

5. A method for producing a ceramic particle, comprising:
providing a core made of magnesium or a magnesium alloy, wherein the core has a diameter of 30-100 μm;
dissolving a calcium salt and a phosphorus salt in a solvent, and adding a chelating agent into the solvent to form a modifying solution; and
adding the core into the modifying solution to form a modification layer on an outer surface of the core in a temperature range of 5-40° C., wherein the modification layer includes calcium and phosphorus.

6. The method for producing the ceramic particle as claimed in claim 5, wherein the calcium salt is calcium nitrate, calcium phosphate, or calcium sulfate.

7. The method for producing the ceramic particle as claimed in claim 5, wherein the phosphorus salt is potassium dihydrogen phosphate, sodium dihydrogen phosphate, or magnesium phosphate.

8. The method for producing the ceramic particle as claimed in claim 5, wherein the chelating agent is edetate disoium or ethylenediamine.
